(19) United States
(12) Patent Application Publication
(10) Pub. No.: US 2004/0111924 A1
(43) Pub. Date: Jun. 17, 2004

(54) Self-stimulating clogs for performing a zonal therapy with changeable stimulation points

(76) Inventor: Raffaele Riccardi, Aprilla (IT)

Correspondence Address: Young & Thompson, Second Floor, 745 South 23rd Street, Arlington, VA, 22202, US

(21) Appl. No.: 10/475,708

(22) PCT Filed: Apr. 23, 2002
(86) PCT No.: PCT/IT02/00264

(51) Int. Cl. A43B 3/12; A61F 5/14

(52) U.S. Cl. 36/141; 36/11.5

(57) ABSTRACT

For performing a zonal therapy on feet without going to experts masseurs, in the plantar of a clog (1) there are inserted small nails having a cap shaped head (2) with a different diameter, which puts out of the plantar itself. Said nails are inserted in points or zones corresponding to organs of the human body which should be stimulated by a reflex action due to the massage resulting from the walking with the clogs.

The present invention refers to self-stimulating clogs with stimulating points changeable according to the point or points of the human body to be stimulated.

Already in ancient times it was known, that some foot points, submitted to pressure, transmit a reflex action to some organs of the human body, not only reducing but also doing away with painful state thereof. Such a zonal stimulation technique is called in the traditional medicine “reflexology”. In practise it is a method of treatment based on the pressure generated by carrying out a manual massage on some foot zones connected by reflex with some organs of the human body, such as for example liver, kidney, head, knees, etc.

Recently, the reflexology has been rediscovered after having ascertain that it really performs a beneficial action on several parts of the human body (see for example the book: “Manual of the reflexology” edited by Pauline Wills, Italian edition of Vallardi I. G.).

However, since the reflexology treatment is based on the massage and on the pressure of the reflexed foot zones, it is necessary that the operator should have a specific knowledge both of the foot anatomy and reflex localization. Obviously, this does not facilitate the diffusion of said traditional technique on a broad scale.

Thus, a purpose of the present invention is to realize a clog apt to stimulate certain points or zones of the foot, which are connected by reflex with some organs of the human body, avoiding thus the necessity to go to expert masseurs, which not always are available or easy to find.

In a further development of this problem, it has been considered to make changeable the points or zones of this clog to submit to the pressure according to the organ or organs of the human body to be stimulated by reflex.

According to the invention, said problems are solved by realizing clogs apt to exert, by walking, a pressure on said foot points or zones by means of interchangeable nails, having a substantially cap shaped head, and thus without sharp projections, which nails can be applied by the user according to its own choice.

Said interchangeable small nails could have, for example, a cap shaped head having a different diameter and can be inserted in suitable points or zones of the clog plantar, which are clearly indicated.

The present invention will be now disclosed in more details with reference to a non-limitative embodiment example shown in the enclosed drawings, in which

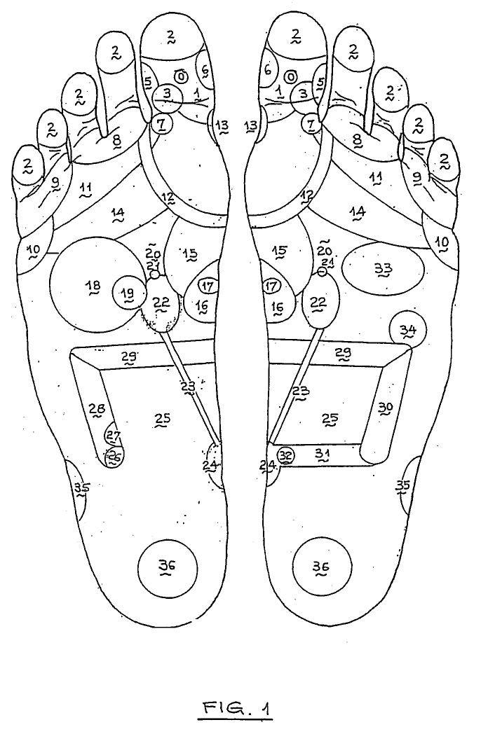

FIG. 1 is a front view of the foot right and left soles, on which there are evidenced the zones to be stimulated according to the traditional knowledge;

FIG. 2 shows a top view of the clog plantar provided with nails, the cap shaped head of which abuts on the plantar itself, in accordance with the foot zones to be stimulated;

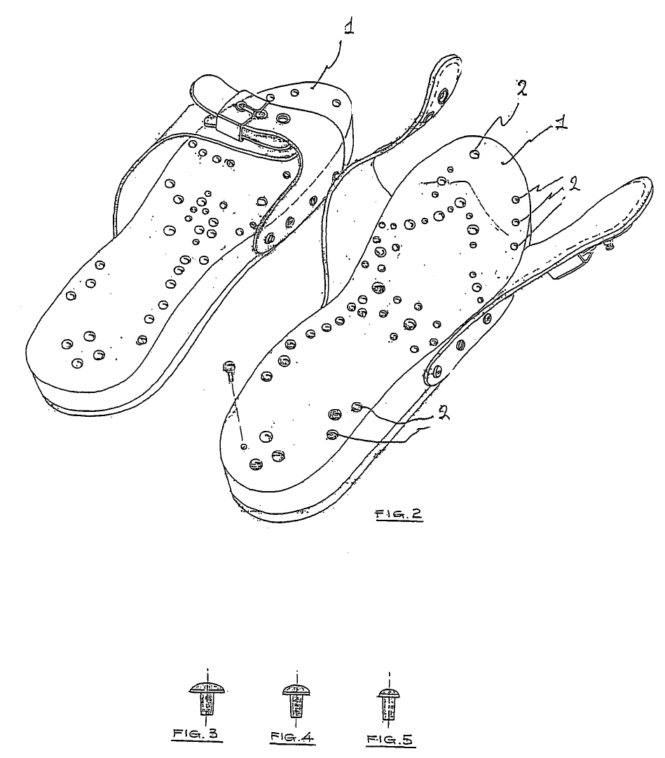

FIGS. 3, 4 and 5 are some examples of nails, which are different as to their sizes and shape of the cap head.

Referring now to the drawings, FIG. 1 shows the known distribution of the stimulating zones of the foot sole, indicated by numerals 1 to 36, which correspond to the organs to be stimulated by reflex, as reported in the following table 1.

TABLE 1

| | | | | | |
|---|---|---|---|---|---|
| 0 | Hypophysis | 13 | Parathyroid | 26 | Appendix |
| | (pituitary) | | | | |
| 1 | Head in general | 14 | Lungs, | 27 | Ileoceleal valve |
| | | | bronchus | | |
| 2 | Frontal sinuses | 15 | Stomach | 28 | Ascending colon |
| 3 | Brainstem, | 16 | Duodenum | 29 | colon transversum |
| | cerebellum | | | | |
| 4 | Epiphysis | 17 | Pancreas | 30 | Descending colon |
| 5 | Temple, | 18 | Liver | 31 | Intestinum rectum |
| | trigeminal | | | | |
| 6 | Nose | 19 | Gall bladder | 32 | Anus |
| 7 | Nape | 20 | Solar plexus | 33 | Heart and blood |
| | | | | | circulation |
| 8 | Eyes | 21 | Suprarenal | 34 | Spleen |
| 9 | Ears | 22 | Kidney | 35 | Knee |
| 10 | Shoulder | 23 | Ureter | 36 | Ovary, Falloppian |
| | | | | | tube/testicles |
| 11 | Trapezium | 24 | Urinary | | |
| | | | bladder | | |
| 12 | Thyroid | 25 | Intestinum | | |
| | | | tenue | | |

In FIG. 2 there are shown the left and right clogs, with stimulating zones, which are shown schematically and only as an example and which correspond to the human body organs indicated in FIG. 1.

In these zones there are applied nails having their heads substantially in form of cap with different diameters, which are apt to stimulate by reflex the corresponding organs of the human body.

Thus the reflex stimulation energy of a painful organ of the human body will be obtained by the choice of the form and/or size of the heads of the nails to be inserted in the clog zone corresponding to this organ, which heads perform by walking more or less energic massage action on the corresponding zone of the foot sole. It is obvious that the nails will be inserted in the corresponding zones of the right and left clogs.

Preferably, as shown in FIGS. 3-5, the clogs are provided with a set of nails having heads in form of more or less pointed cap with a different diameter between 2 and 6 mm, which nails will be inserted in selected zones of the clog, corresponding to the organ to be stimulated, in suitable holes or seats 2 made in the thickness of the clog plantar.

In FIG. 2 only some nails inserted in the corresponding seats 2 of the clog 1 are indicated, whereas the reflex stimulation zones of a human body organ correspond to the respective zones of the foot sole.

The tests carried out on the clog provided with such nails have confirmed their massage and reflex stimulation efficacy, without that the person wearing the clogs complained a troublesome sensation, which apparently the nail heads should have to cause on the foot sole.

The invention has been disclosed with reference to a preferred embodiment thereof, however several changes and modification could be made thereon without going out from the scope of the enclosed claims.

1. A self-stimulating clog with changeable stimulation points, characterized in that it is provided with nails or pins having a cap shaped head (2) of different diameters, which are inserted into the plantar of the clog (1) in points or zones corresponding to human body organs to be stimulated by reflex owing to the massage effect attained by walking with such clogs.

2. A self-stimulating clog according to claim 1, characterized in that the diameter of the cap shaped heads of the nails is between 2 and 5 mm.

3. A self-stimulating clog according to claim 2, characterized in that the degree of stimulation by reflex depends on the diameter and shape of the nail head and on the number of the nails inserted into the zone of the clog plantar intended to perform the massage, by the walking effect, on the corresponding zone of the foot sole and thus on the organ of the human body to be stimulated by reflex action.
